(19) United States
(12) Patent Application Publication
(10) Pub. No.: US 2026/0297677 A1
(43) Pub. Date: Oct. 1, 2026

(54) KIT FOR EARLY DETECTION OF COLORECTAL CANCER DNA USING LATERAL FLOW ASSAY

(71) Applicant: PREDICTIVE AI, Inc., Seongnam-si (KR)

(72) Inventor: Sijung YUN, Seoul (KR)

(21) Appl. No.: 19/092,445

(22) Filed: Mar. 27, 2025

(51) Int. Cl. C12Q 1/6886; B01L 3/00

(52) U.S. Cl. CPC C12Q 1/6886; B01L 3/5023; B01L 2200/0647; B01L 2300/069; B01L 2300/0825

(57) ABSTRACT

A method for detecting colorectal cancer DNA using a lateral flow assay includes: extracting a blood sample (S100); loading the blood sample on a lateral flow assay (LFA) kit (S110); amplifying a specific target circulating tumor DNA (ctDNA) fragment using single nucleotide polymorphism (SNP) typing based on a loop-mediated isothermal amplification (LAMP) device on the kit (S130); allowing the amplified SNP sequence to move along a nitrocellulose membrane and binding to a gold nanoparticle probe to form a DNA-AuNP complex (S140); and color-developing a first probe to be visually detected in the complex forming process (S150), so as to detect whether colorectal cancer mutant DNA exists in the blood sample based on the color development.

### BACKGROUND

### Technical Field

The present disclosure relates to a method for early detection of colorectal cancer DNA using a lateral flow assay (LFA), and more particularly, to a method and a kit for early detection of colorectal cancer DNA using a lateral flow assay capable of rapidly detecting circulating tumor DNA (ctDNA) with a specific genetic mutation in a blood sample by using a loop-mediated isothermal amplification (LAMP) technique.

### Background Art

Colorectal cancer (CRC) still remains a major public health problem in USA, and ranks second among causes of cancer-related deaths. Despite advances in medical technology, the incidence of colorectal cancer is increasing among young adults, which raises the need for innovative screening and diagnostic methods. As current screening methods, fecal immunohistochemical testing (FIT) and colonoscopy are invasive and may delay diagnosis, especially in the younger population. Therefore, there is a critical need for accessible and effective diagnostic tools capable of early detecting colorectal cancer.

With the advent of liquid biopsy technology, a noninvasive alternative to alternate traditional tissue biopsy is enabled for detecting and monitoring of genetic diseases, especially cancer, thereby enabling early detection, real-time monitoring, and personalized treatment. By detecting and analyzing circulating tumor DNA (ctDNA) in blood samples, liquid biopsy may determine a genetic status of a disease in real time. However, widespread adoption of the liquid biopsy is having difficulty requiring high cost, complexity, and a lot of time required by conventional methods.

In addition, while LFA has the potential to democratize access to genetic testing, especially in a resource-constrained environment, due to simplicity, rapidity, and suitability for point-of-care testing, its application has been limited by issues with sensitivity and specificity.

### PRIOR ARTS

### Patent Documents

1. Korean Patent Publication No. 10-2020-0089864 (Method for predicting lung cancer based on blood circulating tumor DNA and circulating tumor cells),
2. Korean Patent Publication No. 10-2023-0172174 (Method for diagnosing and predicting cancer type using single nucleotide variant in cell-free nucleic acid).

### DISCLOSURE

### Technical Problem

Therefore, the present disclosure has been made to solve the problems, and an object of the present disclosure is to provide a method for early detection of colorectal cancer DNA using a lateral flow assay capable of rapidly detecting circulating tumor DNA (ctDNA) with a specific genetic mutation in a blood sample, as an efficient and economical design by developing a paper-based lateral flow assay (LFA) to improve the accessibility of liquid biopsy.

Another object of the present disclosure is to provide a method and a kit for early detection of colorectal cancer DNA using a lateral flow assay capable of precisely and early detecting low-frequency DNA mutations related to rare genetic diseases, cancer, Alzheimer's disease, etc. by utilizing a loop-mediated isothermal amplification (LAMP) technique to increase sensitivity and specificity.

Meanwhile, the technical objects to be achieved in the present disclosure are not limited to the aforementioned technical objects, and other technical objects, which are not mentioned above, will be apparently understood to a person having ordinary skill in the art from the following description.

### Technical Solution

In order to achieve the object, there is provided a method for detecting colorectal cancer DNA using a lateral flow assay including: extracting a blood sample (S100); loading the blood sample on a lateral flow assay (LFA) kit (S110); amplifying a specific target circulating tumor DNA (ctDNA) fragment using single nucleotide polymorphism (SNP) typing based on a loop-mediated isothermal amplification (LAMP) device on the kit (S130); allowing the amplified SNP sequence to move along a nitrocellulose membrane and binding to a gold nanoparticle probe to form a DNA-AuNP complex (S140); and color-developing a first probe to be visually detected in the complex forming process (S150) so as to detect whether colorectal cancer mutant DNA exists in the blood sample based on the color development.

The specific gene may be at least one of KRAS, NRAS, PIK3CA, BRAF, and EGFR genes and a related gene thereof, and the mutation gene to be investigated may be at least one of BRAF V600E, KRAS G12D, KRAS G12V, KRAS G12C, KRAS G12A, KRAS G13D, KRAS A146T, PIK3CA E542K, PIK3CA E545K, and PIK3CA H1047R.

The amplification step may include extending the 5′ end of a Forward Inner Primer (FIP) to an allele of the SNP, and extending the 5′ end of a Backward Inner Primer (BIP) to a complementary allele of the SNP.

The method may further include a step (S160) of indicating whether a second probe is color-developed to operate normally by allowing the extra blood sample to move to a control line.

The method may further include a step (S120) of removing, by a multilayer filter of the kit, impurities such as red blood cells from the blood sample and passing only serum, between the loading step (S110) and the amplification step (S130).

At least one of the LAMP and the multilayer filter may operate with a battery.

The LAMP device may perform amplification at an isothermal temperature of 60 to 65° C.

As another category, the object of the present disclosure as described above may be achieved by a kit for detecting colorectal cancer DNA using a lateral flow assay, including: a loading pad on which a blood sample is loaded; a multilayer filter located at the bottom of the loading pad to remove impurities such as red blood cells from the blood sample and pass only serum; a loop-mediated isothermal amplification (LAMP) device located at the bottom of the multilayer filter to amplify a specific target circulating tumor DNA (ctDNA) fragment in colorectal cancer mutant DNA using single nucleotide polymorphism (SNP) typing; a battery that supplies power to at least one of the LAMP and the multilayer filter; a nitrocellulose membrane located at the bottom of the LAMP device and through which the amplified SNP sequence moves; a first probe of gold nanoparticles that binds to the SNP sequence moving along the nitrocellulose membrane to form a DNA-AuNP complex; and a second probe that indicates whether to be color-developed to operate normally by allowing the extra blood sample to moves to a control line, so as to detect whether colorectal cancer mutant DNA exists in the blood sample based on the color development of the first and second probes.

### Advantageous Effects

According to one embodiment of the present disclosure, point-of-care testing (POCT) is possible at low cost and quickly compared to conventional invasive, expensive, and time-consuming diagnostic methods.

In addition, the LFA used in the present disclosure can early detect colorectal cancer by using the loop-mediated isothermal amplification (LAMP) method to detect specific circulating tumor DNA (ctDNA) and genetic mutations in blood. Such LAMP can amplify target sequences at an isothermal temperature of 60 to 65° C. using a heater operated by an inexpensive battery without requiring a conventional thermal cycler. As a result, there is an advantage of being easy to move and simply maintained.

In addition, the kit of the present disclosure is operated by a battery, so that LAMP and a multilayer filter can quickly detect genetic mutations at low cost.

However, effects which can be obtained in the present disclosure are not limited to the aforementioned effects and other unmentioned effects will be clearly understood by those skilled in the art from the following description.

### DESCRIPTION OF DRAWINGS

The accompanying drawings of this specification exemplify a preferred embodiment of the present disclosure, the spirit of the present disclosure will be more clearly understood from the following detailed description taken in conjunction with the accompanying drawings, and thus it will be understood that the present disclosure is not limited to only contents illustrated in the accompanying drawings:

FIG. 1 is a graph showing 10 mutant DNAs identified in 18 colorectal cancer (CRC) studies according to cBioPortal that may be used for early detection of colorectal cancer DNA using a lateral flow assay of the present disclosure;

FIG. 2 is a schematic perspective view of a lateral flow assay (LFA) kit proposed for ctDNA detection according to the present disclosure;

FIG. 3 is a schematic diagram of LAMP-based SNP typing according to the present disclosure;

FIG. 4 shows an example of a 601 bp long ctDNA sequence including a SNP allele in the middle (SNP from T to A is indicated in bold letters);

FIG. 5 shows a screen capture of the output of PrimerExplorer;

FIG. 6 shows an example of a modified LAMP primer sequence for amplifying ctDNA with a SNP allele; and

FIG. 7 shows an example of a final LAMP primer sequence for amplifying a BRAF_V600E ctDNA fragment.

### MODES

Hereinafter, embodiments of the present disclosure will be described in detail so as to be easily implemented by those skilled in the art, with reference to the accompanying drawings. However, the description of the present disclosure is merely embodiments for the structural and functional description and the scope of the present disclosure should not be construed as being limited by embodiments described in a text. That is, since the embodiments may be variously changed and have various forms, the scope of the present disclosure should be understood to include equivalents capable of realizing the technical spirit. Further, it should be understood that since a specific embodiment should include all objects or effects proposed in the present disclosure or include only the effect, the scope of the present disclosure is limited by the object or effect.

Meanings of terms described in the present disclosure should be understood as follows.

The terms “first”, “second”, and the like are used to distinguish a component from the other component, but the scope of the present disclosure should not be construed to be limited by the terms. For example, a first component may be referred to as a second component, and similarly, the second component may be referred to as the first component. It should be understood that, when it is described that a component is “connected to” the other component, the component may be directly connected to the other component or another component may be present therebetween. In contrast, it should be understood that when it is described that a component is “directly connected to” the other component, another component is not present therebetween. Meanwhile, other expressions describing the relationship between the components, that is, expressions such as “between” and “directly between” or “adjacent to” and “directly adjacent to” should be similarly interpreted.

It is to be understood that the singular expression include a plurality of expressions unless the context clearly indicates otherwise, and it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise defined, all terms used herein have the same meanings as those generally understood by those skilled in the art. Terms which are defined in a generally used dictionary should be interpreted to have the same meaning as the meaning in the context of the related art, and are not interpreted as an ideal meaning or excessively formal meanings unless otherwise clearly defined in the present disclosure.

### EXAMPLES

Hereinafter, the configurations of preferred Examples will be described in detail with reference to accompanying drawings. First, a lateral flow assay (LFA) may increase the accessibility of genetic testing in an environment (e.g., on-site) with limited surrounding conditions due to its simplicity, speed, and suitability for point-of-care testing. However, its application has been limited due to issues with the sensitivity and specificity of LFA.

### Identification and Profiling of Colorectal Cancer Biomarkers

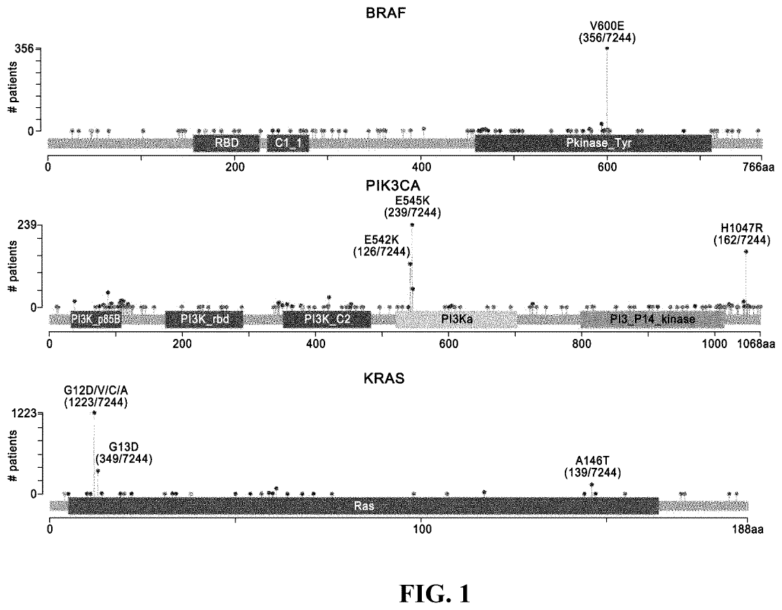

First, 74 mutations in five genes of KRAS, NRAS, PIK3CA, BRAF, and EGFR were targeted, which were consistent with Agena Bioscience's UltraSEEK® Colon Pane. In initial analysis, 10 somatic mutations in the BRAF, KRAS, and PIK3CA genes were identified to be associated with colorectal cancer. These three genes had particularly high importance in colorectal cancer. FIG. 1 is a graph showing the frequency of each of 10 mutations associated with colorectal and colonic adenocarcinomas in 18 studies published in cBioPortal. These studies included 7,420 samples collected from a total of 7,244 patients.

Specifically, one mutation in the BRAF gene, six mutations in the KRAS gene, and three mutations in the PIK3CA gene were identified. The 10 selected mutations, BRAF V600E, KRAS G12D, KRAS G12V, KRAS G12C, KRAS G12A, KRAS G13D, KRAS A146T, PIK3CA E542K, PIK3CA E545K, and PIK3CA H1047R, were known to significantly affect the progression of colorectal cancer and patient responses to various treatments. The inclusion of these major mutations provides an excellent opportunity for early detection and personalized treatment strategies.

### BRAF-V600E

A BRAF gene encodes a serine/threonine protein kinase and plays a key role in the regulation of a mitogen-activated protein kinase (MAPK) pathway. BRAF mutations promote cell proliferation and evasion of apoptosis by continuously activating the kinase. In particular, a V600E mutation causes abnormal hyperactivity in a MAPK pathway regardless of RAS. The V600E mutation was located in exon 15 and resulted in substitution of valine (V) to glutamic acid (E) at position 600. Approximately 10% of colorectal cancer (CRC) patients exhibit BRAF mutations, and V600E is a major mutation, accounting for 95% of all BRAF mutations. In addition, approximately 30% of the BRAF mutations are consistent with a CpG island methylator phenotype (CIMP) and high-level microsatellite instability (MSI) due to methylation of an MLH1 promoter. These molecular characteristics form a specific subtype of colorectal cancer (CRC) with unique clinical and pathologic features.

The BRAF V600E mutation is associated with resistance to anti-Epidermal Growth Factor Receptor (anti-EGFR) monoclonal antibodies and overall poor prognosis. This mutation causes aggressive tumor behavior and is associated with poor survival rate compared to BRAF wild-type CRC. Patients with metastatic colorectal cancer (CRC) often develop resistance to EGFR and BRAF inhibitors. Current FDA-approved treatments for CRC patients with BRAF V600E mutation include a combined therapy of an RAF-targeting inhibitor, Encorafenib and an anti-EGFR antibody, Cetuximab. Although the combined therapy has shown improved efficacy compared to a monotherapy, it is still difficult to manage BRAF-mutation CRC due to the development of resistance and tumor heterogeneity.

Despite the development, there is still a difficulty in the clinical management of BRAF-mutation CRC. Personalized treatment approaches guided by molecular profiling are gradually recognized as important for improving outcomes in this patient group. Current research focuses on developing novel therapeutic strategies, particularly combined therapies targeting multiple pathways and resistance mechanisms. Integrating clinical practice of precision medicine offers hope for more effective treatment responses by providing treatments according to the genetic and molecular characteristics of each patient's tumor.

### KRAS—G12V, G12C, G12A, G13D, A146T

A KRAS gene encodes a GTP/guanosine diphosphate (GDP)-binding protein and plays a critical role in regulating rapidly progressive fibrosarcoma (RAF)-mitogen-activated protein kinase (MAPK), MEK-extracellular signal-regulated kinase (ERK), and phosphoinositol 3-kinase (PI3K)-protein kinase B (AKT)-mammalian target of rapamycin (mTOR) pathways. KRAS mutations have a significant effect on treatment responses. In particular, these mutations affect the effect of an anti-epidermal growth factor receptor (EGFR) therapy, and show better treatment responses to patients with wild-type KRAS genes. Detection of KRAS mutations is considered essential before starting anti-EGFR antibody therapy. In addition, when BRAF mutations coexist with KRAS mutations, anti-EGFR therapy response may be further complicated, especially after primary treatment.

Mutations in a RAS protein group are found in approximately half of all CRC cases, which are particularly noteworthy. Mutations in both KRAS and NRAS have similar effects on downstream growth factor signaling, and harmful mutations in these proteins cause continuous growth signals in cells even in the absence of upstream cell surface signals by EGFR or TGFα. Common mutations in KRAS and NRAS occur at codons 12 and 13, and affect the ability of proteins to hydrolyze and inactivate bound GTP, resulting in a permanently activated RAS protein state that does not respond even to changes in upstream signaling induced by anti-EGFR therapy. According to a comprehensive study of the COSMIC database, it was identified that in 18,376(34 %) of 53,978 colorectal cancer samples, KRAS had mutations at codons 12 and 13. Within codon 12, KRAS G12D is the most common, while G12V is the second most common and is associated with a higher risk of death. NRAS mutations were less common and occurred in 150 (2%) of 8,669 colorectal cancer samples at codons 12 and 13, but the mechanism was similar to that of analogous KRAS mutations. According to the study, KRAS and NRAS mutations are likely to be mutually exclusive at these positions. In addition, KRAS mutation-positive results were also found to be associated with the severity of metastasis. The possibility of KRAS mutations increased as each stage of the Dukes'staging system increased, and approximately two-thirds of severe metastatic (stage D) cases were mutated, whereas only one-quarter of localized (stage A) cases were mutated. This suggests that liquid biopsy diagnosis may be used as a broader indicator of disease severity.

### PIK3CA—E542K, E545K, H1047R

A PIK3CA gene encodes a catalytic subunit of PI3-kinase, and regulates various cellular processes such as cell growth, proliferation, and survival, as a key enzyme involved in a PI3K/AKT signaling pathway. PIK3CA mutations result in continuous activation of a PI3K pathway, leading to cell growth and tumor progression. These mutations occur in 10 to 20% of colorectal cancer (CRC) patients, and most often in exons 9 and 20.

E542K and E545K mutations include mutations which are located in exon 9 and substituted from glutamic acid (E) to lysine (K) in a helical domain of the protein. These mutations cause continuous activation of PIK3CA by losing p85-mediated inhibition of catalytic activity. A H1047R mutation includes mutations which are located in exon 20 and substituted from histidine (H) to arginine (R) in a kinase domain of the protein. This mutation exposes the catalytic loop to the cell membrane to continuously activate the catalytic subunit of PI3K. The exposure further enhances the catalytic activity of PI3K to promote oncogenic signaling. All three mutations E542K, E545K, and H1047R are considered as hotspot mutations that frequently occur in colorectal cancer. These mutations not only promote tumorigenesis, but are also associated with treatment resistance. In particular, these mutations may impart resistance to treatment such as Fulvestrant, an estrogen receptor antagonist used in cancer hormone therapy.

In addition, the presence of PIK3CA mutations may affect the clinical management of colorectal cancer patients, which may provide information on combined therapies to overcome resistance and improve treatment efficacy.

### LFA Assay

A paper-based lateral flow assay (LFA) according to the present disclosure was designed to detect DNA fragments with higher sensitivity and specificity than conventional assays. The LFA utilizes a loop-mediated isothermal amplification (LAMP) method to enhance the ability to detect specific circulating tumor DNA (ctDNA) and genetic mutations in blood. DNA amplification was critical to increase the sensitivity and specificity of the assay, but conventional thermal cyclers required expensive laboratory equipment and were impractical for field use. The LAMP overcomes this limitation by enabling isothermal amplification of target sequences at 60 to 65° C. This reaction may be performed using a simple, inexpensive, battery-operated heater.

In addition, the present disclosure optimizes ctDNA extraction by innovating a collection procedure of blood samples. By refining this process, it is possible to more effectively detect low-frequency DNA mutations that are helpful in the diagnosis and monitoring of genetic diseases including cancer and Alzheimer's disease. The LFA includes a multilayer filter that removes unnecessary components such as red blood cells and separates serum containing ctDNA.

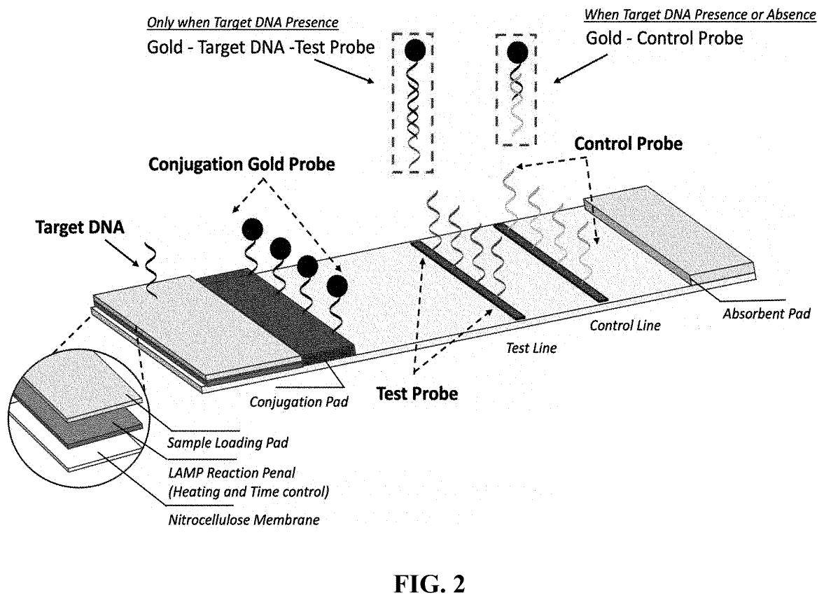

FIG. 2 is a schematic perspective view of a paper-based lateral flow assay (LFA) kit proposed for ctDNA detection according to the present disclosure. As shown in FIG. 2, after loading a blood sample, a multilayer filter in a sample loading pad removes impurities to leave only serum for ctDNA detection. Specific target ctDNA fragments are amplified using LAMP-based single nucleotide polymorphism (SNP) typing. The 5′ ends of Forward Inner Primer (FIP) and Backward Inner Primer (BIP) were designed to be extended to an SNP allele and its complementary allele, respectively.

In an embodiment of the present disclosure, a LAMP-based signal amplification method is used to enhance the sensitivity and specificity of the assay. After this optimization work is completed, in a subsequent validation step, clinical evaluation of the performance of the assay is performed.

### Goal 1—Optimization of Paper-Based Lateral Flow Assay for Enhanced ctDNA Detection

Goal 1 is to develop a kit prototype of a paper-based lateral flow assay (LFA) capable of detecting circulating tumor DNA (ctDNA) and specific DNA mutations in blood with enhanced sensitivity and specificity by utilizing LAMP technology.

### Primers for SNP-Specific LAMP Amplification

Here, it is targeted to amplify ctDNA fragments having specific SNP rather than wild type. This is called LAMP-based SNP typing. It is a key that a 5′ end of the Forward Inner Primer (FIP) extends to an SNP allele, and a 5′ end of the Backward Inner Primer (BIP) extends to the complementary allele of the SNP.

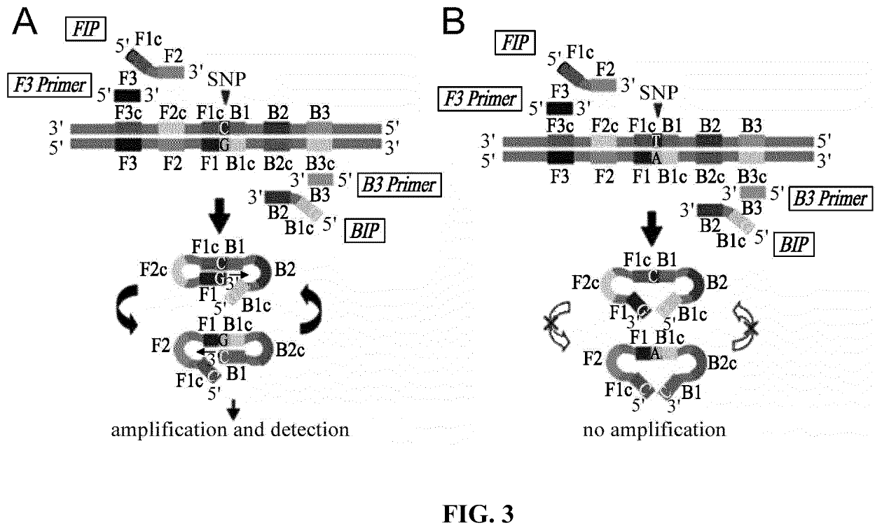

FIG. 3 is a schematic diagram of LAMP-based SNP typing according to the present disclosure. An MEVB vaccine strain in the first drawing is a schematic diagram of a DNA fragment that amplifies only an SNP allele, and a wild-type allele is not amplified in a wild-type MEV strain in the second drawing.

Thereafter, the amplified SNP sequence moves along a nitrocellulose membrane and binds to a gold nanoparticle probe to form a DNA-AuNP complex. These nanoparticles are hybridized to emit strong red fluorescence and develop color, which may be confirmed to the naked eye and is ideal for on-site diagnosis. If a target DNA is present, the target DNA hybridizes with a complementary sequence in a first probe of a test line to generate a red signal. The extra blood sample continuously moves to a control line to allow a second probe to color-develop a second red signal. As a result, it may be confirmed that the analysis is working properly.

For example, technical details of an LAMP primer sequence design were shown to amplify a ctDNA fragment mutated from T to A in a BRAF gene. This mutation is transformed from valine (V) of amino acid at position 600 to glutamic acid (E) (NM_.0043336(BRAF):c.1799T>A (p. Val600Glu)).

### Goal 1—Step 1: Obtainment of 601 bp Long ctDNA Fragment Sequence With SNP Sequence in the Middle

In a ClinVar item for this mutation, a coordinate in the reference genome GRCh38 is indicated as chr7:140753336. Therefore, chr 7:140753036-140753636 is a 601 bp long interval with SNP in the middle. Since a gene is located in a reverse chain, a reverse complement option is selected using a UCSC genome browser and then a FASTA file with a wild-type allele in the middle is obtained. Then, T′ is manually edited to ‘A’ to obtain a ctDNA fragment FASTA file containing the SNP allele.

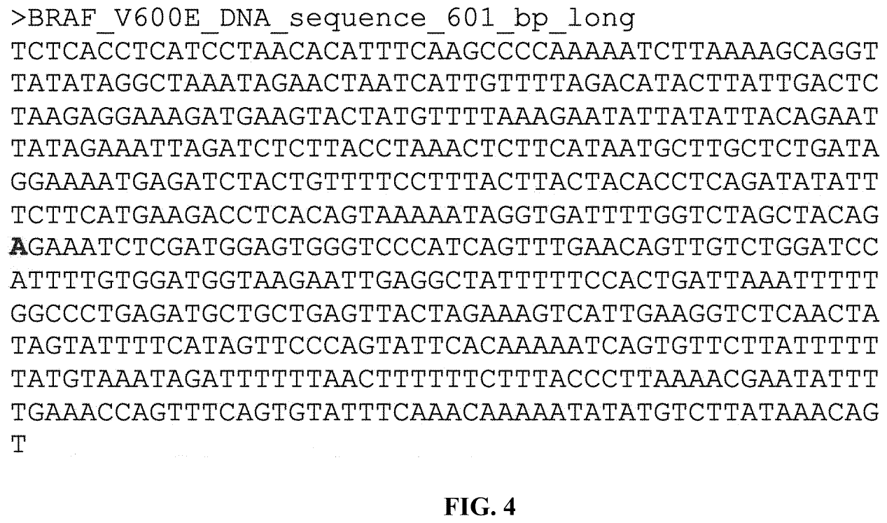

FIG. 4 shows an example of a 601 bp long ctDNA sequence including a SNP allele in the middle (SNP from T to A is indicated in bold letters).

### Goal 1—Step 2: Execution of PrimerExplorer on LAMP Primer Design Website

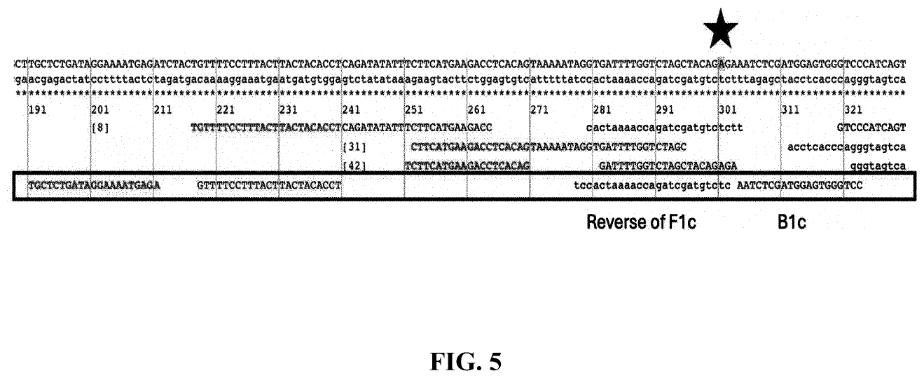

Next, PrimerExplorer version 5, a website that generated LAMP primers, was used. The FASTA file above was used as an input, and four candidate LAMP primer sets were generated using a set of parameters set to automatic judgment. FIG. 5 is a screen capture of the output of PrimerExplorer.

In FIG. 5, the first line is an input FASTA sequence with a SNP allele ‘A’ marked with an asterisk. The second line is the reverse complement sequence. The last line, marked with a box, is a candidate sequence aligned with the input FASTA.

### Goal 1—Step 3: Modification of Primer Sequence Obtained From PrimerExplorer to Amplify Non-Wild Type SNP Alleles

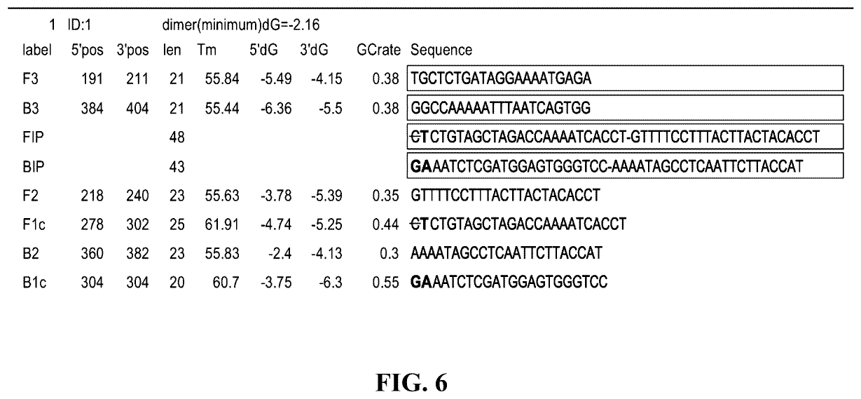

To amplify only ctDNA with non-wild type SNP alleles, a reverse direction of F1c should end with the SNP allele and B1c should start with the SNP allele. The sequence was manually modified for SNP amplification as shown in FIG. 5. FIG. 6 shows an example of a modified LAMP primer sequence for amplifying ctDNA with a SNP allele. As shown in FIG. 6, final four LAMP primer sequences are shown to amplify only a ctDNA fragment with a mutation from T to A in the BRAF gene.

A middle cut line in FIG. 6 represents a deleted base (C), and bases (GA) in bold font are inserted bases. Bases (T) in bold font represent the SNP alleles. The 5′-end of FIP is a SNP allele, and the 5′-end of BIP is a complementary allele of SNP.

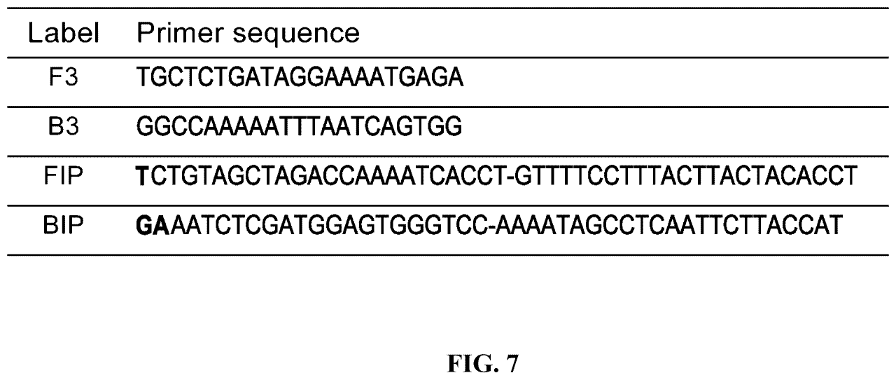

FIG. 7 shows an example of a final LAMP primer sequence for amplifying a BRAF_V600E ctDNA fragment.

### Goal 2—Systematic Analysis of Sensitivity and Specificity of Lateral Flow Assay

Goal 2 aims to comprehensively evaluate the sensitivity and specificity of a lateral flow assay (LFA) for detecting circulating tumor DNA (ctDNA). The sensitivity, specificity, and an area under the curve (AUC) for a receiver operating characteristic (ROC) are calculated, and these values are based on 90% or higher.

Examples of the present disclosure do not use human samples or real human data, but instead, artificially synthesized DNA fragments having the above-mentioned sequences were injected at controlled concentrations, and the sensitivity and specificity were measured.

After a prototype was fabricated, sensitivity (True Positive Rate, TRP) and specificity analysis were performed to evaluate the performance of the LFA. Each criterion showed an effect of 90% or more, and the AUC (Area Under the Curve) value of the ROC (Receiver Operating Curve) was 0.9 or higher.

As described above, the detailed description of the preferred embodiments of the present disclosure disclosed above has been provided so as for those skilled in the art to implement and execute the present disclosure. While the present disclosure has been described with reference to the preferred embodiments, it will be understood to those skilled in the art that various changes and modifications of the present disclosure may be made without departing from the scope of the present disclosure. For example, those skilled in the art may use respective components disclosed in the embodiments by combining the respective components with each other. Therefore, the present disclosure is not limited to the embodiments described herein, but intends to grant the widest range which is coherent with the principles and new features disclosed herein.

The present disclosure may be embodied in other specific forms without departing from the spirit and essential characteristics of the present disclosure. Accordingly, the aforementioned detailed description should not be construed as restrictive in all terms and should be exemplarily considered. The scope of the present disclosure should be determined by rational construing of the appended claims and all modifications within an equivalent scope of the present disclosure are included in the scope of the present disclosure. The present disclosure is not limited to the embodiments described herein, but intends to grant the widest range which is coherent with the principles and new features presented herein. Further, the claims that are not expressly cited in the claims are combined to form an embodiment or be included in a new claim by an amendment after the application.

This invention was made with government support under Project Unique Number RS-2024-00511676, funded by the Ministry of SMEs and Startups through the Korea Technology and Information Promotion Agency for SMEs (TIPA). The research was conducted as part of the Startup Growth Technology Development Project (TIPS), under the research project titled “Development of a POCT Device for Early Diagnosis, Recurrence, and Drug Resistance Monitoring of Hematologic Cancers Using Paper-based LAMP-LFA Technology for DNA Biomarker Detection and a DNA-based Human Digital Twin AI Integrated Platform Specialized in Hematologic Cancers.” The contribution rate of the government support was 100%, and the lead organization for the project was PREDICTIVE AI, Inc.

1. A kit for detecting colorectal cancer DNA using a lateral flow assay, comprising:
a loading pad on which a blood sample is loaded;
a multilayer filter located at the bottom of the loading pad to remove impurities such as red blood cells from the blood sample and pass only serum;
a loop-mediated isothermal amplification (LAMP) device located at the bottom of the multilayer filter to amplify a specific target circulating tumor DNA (ctDNA) fragment in colorectal cancer mutant DNA using single nucleotide polymorphism (SNP) typing;
a nitrocellulose membrane located at the bottom of the LAMP device and through which the amplified SNP sequence moves;
a first probe of gold nanoparticles that binds to the SNP sequence moving along the nitrocellulose membrane to form a DNA-AuNP complex; and
a second probe that indicates whether to be color-developed to operate normally by allowing the extra blood sample to move to a control line, so as to detect whether colorectal cancer DNA exists in the blood sample based on the color-development of the first and second probes.

2. The kit for detecting colorectal cancer DNA using the lateral flow assay of claim 1, wherein a specific gene in the specific target circulating tumor DNA is at least one of KRAS, NRAS, PIK3CA, BRAF, and EGFR genes and a mutant gene thereof.

3. The kit for detecting colorectal cancer DNA using the lateral flow assay of claim 2, wherein the specific gene is at least one of KRAS, PIK3CA, and BRAF genes and a related gene thereof.

4. The kit for detecting colorectal cancer DNA using the lateral flow assay of claim 2, wherein the mutant is at least one of BRAF V600E, KRAS G12D, KRAS G12V, KRAS G12C, KRAS G12A, KRAS G13D, KRAS A146T, PIK3CA E542K, PIK3CA E545K, and PIK3CA H1047R.

5. The kit for detecting colorectal cancer DNA using the lateral flow assay of claim 1, wherein the LAMP device performs extending the 5′ end of a Forward Inner Primer (FIP) to an allele of the SNP, and extending the 5′ end of a Backward Inner Primer (BIP) to a complementary allele of the SNP.

6. The kit for detecting colorectal cancer DNA using the lateral flow assay of claim 1, wherein the color-development is red or blue.

7. The kit for detecting colorectal cancer DNA using the lateral flow assay of claim 1, further comprising:
a battery that supplies power to at least one of the LAMP and the multilayer filter.

8. The kit for detecting colorectal cancer DNA using the lateral flow assay of claim 1, wherein the LAMP device performs amplification at an isothermal temperature of 60 to 65°C.
